(19) United States
(12) Patent Application Publication
(10) Pub. No.: US 2010/0222670 A1
(43) Pub. Date: Sep. 2, 2010

(54) DEVICE FOR MEASURING AND METHOD FOR ANALYSING GASTROINTESTINAL MOTILITY

(76) Inventors: Michel Demierre, Semsales (CH); Vincent Schlageter, Lausanne (CH); Pavel Kucera, Lausanne (CH)

Correspondence Address: NIXON & VANDERHYE, PC, 901 NORTH GLEBE ROAD, 11TH FLOOR, ARLINGTON, VA, 22203, US

(21) Appl. No.: 12/681,712

(22) PCT Filed: Oct. 6, 2008
(86) PCT No.: PCT/IB08/54091

§ 371 Date: Apr. 5, 2010

(30) Foreign Application Priority Data
Oct. 4, 2007 (EP) PCT/EP2007/060561

(51) Int. Cl. A61B 5/05

(52) U.S. Cl. 600/424

(57) ABSTRACT

The invention relates to a device for measuring the intestinal motility, that comprises a solid marker intended for travelling along the entire digestive system of a living being, or a portion thereof, a detector including means for detecting the position and the orientation of the marker, means for measuring the intestinal motility connected to the detector, wherein the marker and the detector includes transmission means and reception means for transmitting and receiving electromagnetic signals that ensure a bi-directional communication between the marker and the detector. The invention also relates to the use of the above-mentioned device.

### FIELD OF THE INVENTION

This invention relates to the field of gastro-intestinal motility analysis.

### BACKGROUND OF THE INVENTION

As far as anatomy is concerned, the digestive system compares to a tube that begins with the mouth and ends with the anus and is segmented by sphincters. These segments are: the oral cavity, esophagus, stomach, small intestine (subdivided in duodenum, jejunum, and ileum), colon (subdivided in cecum and ascending, transverse, descending, and sigmoid),

Gastro-intestinal (“GI”) motility is the mechanical activity of the digestive system. Together with secretion and absorption, it is one of the main digestive functions. GI motility is involved in mechanical fragmentation, mixing with secretions, homogenization, peristalsis (contents propulsion), functional partition between the different segments by high pressure areas, storage and excretion of stools.

GI motility is based on contraction of smooth muscles layers. When these contractions, longitudinal and circular, are distributed along the tube and spatially and temporally arranged, the outcome is a complex motility pattern specific for each GI segment.

This motility originates in non-neural mechanisms (rhythmic variations of the electric potential of smooth muscle cells, or “slow waves”, generated by pacemaker cells, or cells of Cajal) as well as neural mechanisms (enteric nervous system ENS innervation, central nervous system CNS communication). CNS allows an answer to various stimuli, such as mechanical, chemical, electrical, and thermic stimuli. Sympathetic and parasympathetic communication gives an unconscious perception of the state of the digestive system to the brain which affects it in return. For instance, a stressful situation may trigger a sudden increase of the peristalsis (diarrhea) or quite the opposite a strong inhibition (constipation).

Motility disorders are of organic or functional origin and take numerous forms, such as dysphasia, dyspepsia, gastroparesis, ileus, irritable bowel syndrome, diarrhea, chronic constipation, etc.

The diagnosis of these pathologies is based among others on intraluminal manometry and pH-metry and imaging techniques (endoscopy, X-rays, scintigraphy, CT-scan). These conventional techniques are often invasive and unpleasant for the patient, moreover they provide only limited information for the diagnosis of functional troubles. The information is mostly limited to proximal and distal segments and/or limited to a short period of time. Today, there is no technique providing a real analysis of the motility, in particular of the dynamics of the displacement of the luminal content.

These limitations on diagnosis also represent an obstacle to an effective treatment, because it is difficult to determine with sufficient accuracy the effect of drugs, of surgery or of other therapies.

Less invasive means based on ingested foreign body (e.g. smart pills) exist. An imaging pill (see patent application EP 0 667 115 A1) complements standard endoscopy, in particular for the small intestine. Other pills measure pH and/or pressure (see for example patent application US 2008/004547).

A common issue for all state of the art pills lies in the impossibility to localize accurately their progression along the digestive tube, to know in which segment of the digestive tract they are located. Knowing their approximate position with respect to an external landmark is not sufficient.

In the text below, we use the term “localization” or “to localize” to address the progression along the digestive tube. For instance, the marker is localized in the jejunum 10 cm after the angle of Treitz. On the contrary, if we talk about the “position” of the marker, we mean the coordinates, e.g. Cartesian, of the marker with respect to the detector.

Techniques based on the ingestion and tracking the position of one or several markers have already been proposed. At first based on ferromagnetic markers (see for example patent application WO 2004/014225) or tracers (powder or liquid, requiring expensive sensors “SQUIDs” operating in a controlled environment), it has also been suggested to use an alternative magnetic field in order to avoid limitations due to the earth magnetic field (WO 2004/014225).

However, these recent techniques contain several drawbacks. They are characterized in particular by too large a power consumption. But, an optimal management of the energy available in a small size capsule is necessary, in particular if it aims at the examination of the digestive tube as a whole.

Moreover, methods to analyze GI motility are lacking, in particular concerning tracking of markers so as to provide the physician simultaneously with information about the progression (transit) and about the local activity.

Therefore, there is a need for a system enabling long term and repeatable examinations, minimally invasive, providing more relevant information concerning the digestive motility, the whole with an optimum management of the energy needed by the device to operate.

### SUMMARY OF THE INVENTION

The present invention offers a solution to the problems explained in the previous section,

It concerns, in the first place, a device for monitoring GI motility comprising of at least a solid marker (pill, suppository, etc . . . ) intended for covering all or part of the digestive system of a living being, a detector comprising means of detecting position and orientation of the aforementioned marker, means of measuring the GI motility connected to the detector. The marker or markers as well as the detector comprising each means of emission and reception of electromagnetic signals intended for providing a bidirectional communication between the marker or markers and the detector.

Several embodiments of the invention are described in the claims.

Advantageously, the marker comes as a pill containing the means of reception and emission. In the text below, one will use most often the word “pill” to illustrate this preferred embodiment of the marker.

According to one embodiment of the invention, the pill contains coils functioning as both an emitter and receiver of electromagnetic signals. The bidirectional communication achieved this way between the marker and the detector offer several advantages as one will see further on.

Advantageously, the detector also includes one or several coils.

Thanks to its means of emission, the detector can send signals to the marker(s).

These signals can be used for different purposes, in particular the selection of a marker to communicate with each marker separately, the transition from a standby mode to an emission mode, the variation of the emission power trying to optimize the energy consumption, the synchronization of the marker emission with the detector listening, the synchronization of the marker listening with the detector emission in order to optimize the marker autonomy, as well as the synchronization of the phase of the signal emitted by the marker in order to improve the demodulation (amplitude measurement).

Markers may be in different working modes, in order to optimize their energy consumption. Advantageously, they have at least three working modes: turned off, standby, and emission. When turned off, the marker is passive and consumes no or almost no energy. In standby mode, the circuits necessary for its operation during the examination are active. In the emission mode, a magnetic field enabling the calculation of its position is emitted.

Transition from the turned off mode to the standby mode, so called activation, occurs preferably at the time of initialization, normally when the marker is ingested. At the time of initialization, different parameters varying from a marker to another may also be calibrated.

Depending on the chosen activation method, an activation coil is necessary. Preferably, this coil also belongs to the detector, but it may also be part of an independent device. It enables an energy transfer which may be followed by an information transfer via the marker coils.

Transition from the standby mode to the emission mode is preferably made with the synchronization coil, and subsequently the marker reverts to the standby mode.

When current is initiated in the emitting coils, a marker generates two or three magnetic orthogonal dipoles depending on the design. Since the shape of the emitted magnetic field is known—close to an ideal dipole—it is possible to calculate the position of the emitter with 6 degrees of freedom. For that, the detector measures the amplitude and the phase of the magnetic signal at different locations and/or in different orientations. This layout greatly improves the convergence of the algorithm for extracting the position, as compared to a pill generating a single dipole.

In addition to the 6 degrees of freedom of the position and orientation of the pill, the emitted magnetic momentum may also be used to transmit other information coming from a sensor in the marker by modulation of the amplitude, the phase or the frequency of the emitted magnetic momentum.

The generated magnetic momentum is alternating—close to a sine wave. To save energy, the signal is not emitted continuously but intermittently with a low duty cycle. Besides the energy conservation, a short emission is preferable in order to guarantee a constant position and especially a constant orientation of the pill during the whole emission period.

According to one embodiment of the invention, the detector contains receiving coils enabling an inductive measure of the amplitude of the magnetic signals from the markers. At least 2 or 3 receiving coils (depending on the number of emitting coils) are needed to recalculate the position and orientation of the pills. Redundancy, obtained with a larger number or receiving coils, is desired to increase the accuracy and the volume where the tracking is possible. Receiving coils are either oriented in the same direction or in different directions, for example so as to measure the three components of the field at a given location (3D coil).

Relative position and orientation of each receiving coil must be known with accuracy. If this requirement is not fulfilled, it is not possible to obtain sufficient information to study motility. This is made possible, for instance, with a rigid support on which the coils are fixed, or it may be a bendable support whose change of shape can be accurately measured (for instance one or two articulations including an angular sensor).

Advantageously, the detector is placed close to the abdomen, for instance inserted in a harness. A fixed device is also possible and sufficient to study the upper digestive tract, for instance gastric emptying. A fixed position for the detector enables minimizes artifacts due to movements of the subject and gives a better standardization of the recordings. In this case, the detector is built-into a chair or a bed.

Movements of the subject as well as his breathing generate movement artifacts, i.e. displacement of viscera with respect to the detector. In order to detect these artifacts, to filter them out, to estimate the quality of the recordings and to determine the type of activity of the subject, additional sensors are used in the detector. Collected information may be used for modifying emission parameters of the pills as well as for data processing. Other physiological records useful for data interpretation may also be collected in the detector.

Visualization of the raw trajectory offers already many observation possibilities.

However, post-processing of raw data is desired in order to reduce artifacts, to extract the most relevant information while reducing the amount of data, and to quantify motility (motility indices).

A suggested approach consists of several steps. The first one is to filter out artifacts as much as possible. The number and amplitude of artifactual movements may be at least as important as the movements due to digestive motility, which is also a problem in other examination techniques such as manometry. Generally, two types of artifacts have to be detected and filtered out: technical artifacts and movement artifacts. A technical artifact occurs when the position measured by the detector is not the real position (e.g.: if the magnetic measurement is noisy, the algorithm may hesitate between two positions). A movement artifact occurs when the detector moves with respect to the viscera, i.e. either a movement of the detector with respect to the anatomical landmarks, or a movement of the viscera with respect to the anatomical landmarks (e.g.: breathing of the subject, movements while walking).

Once the artifacts have been detected, they are separated from the raw trajectory to obtain the corrected trajectory. They also give the quality of the recording, enabling, for instance, to ignore some parts of the recording.

A first analysis enables to partition the data, that is to subdivide and to sort them according to the segment of the digestive tube involved and also according to the type of displacement. The different segments are the esophagus, stomach, small intestine, and large intestine, each one can be subdivided in sub-segments (for example, two sub-segments for the stomach proximal/distal, three for the small intestine duodenum/jejunum/ileum, five for the large intestine: cecum-ascending colon/hepatic angle-first part of transverse colon/ last part of transverse colon-splenic angle/ descending/sigmoid colon). There is typically three types of displacement, namely periods without net displacement, with slow displacements and with fast displacements.

Data segmentation aims, for the next steps, to fit the analysis algorithm with the current data segment. Thus, for instance, the part of the skeleton corresponding to a data segment when the maker is in the stomach without net displacement will not be calculated like the part corresponding to a fast displacement in the colon.

Analysis of rhythmic activity and tracking of frequencies specific to each digestive segment, breaking down the trajectory in net displacement on one side and back-and-forth movements on the other side, among other information, permit this data segmentation.

The next step consists of calculating the anatomic trajectory, i.e. the middle line of the digestive tube called hereafter the skeleton. Once the skeleton is available, 3D data are projected onto this line. The resulting ID trajectory yields a much simpler analysis of the dynamics of the progression. Of course, the orientation of the pill is also taken into account, in particular the relative angle between the pill and the skeleton.

At this stage, it is possible to calculate different indices of motility and defining the activity, of each segment of the digestive tract. In particular, displacement indices and local activity indices are defined.

One or several pills are ingested and data are recorded following a protocol selected to fit the pathology. This protocol defines in particular the pills ingestion time (for instance each morning at waking up), meals time and periods of waking state and periods of motionlessness enabling higher quality recordings. This protocol may also include therapeutic acts, such as drugs intake, massages, or other acts that stimulate the digestive system.

Significant interindividual variations exist in normal patient, giving large standard deviation for normal values. To avoid this problem, the patient may be used as his own control (for instance, two recordings with and without drugs).

The response to a state change, defined in the protocol, is an alternative to the above method. In this case, the change is made during the recording. For instance, a search is made for the presence or the absence of a gastro-colic reflex following food intake, or an activity increase following wake up or drug intake.

Tracking several markers (e.g. five) sequentially offers many possibilities. In particular to study coordination between different segments of the digestive tract (reflexes, correlations, activity wave propagation), or for statistical reason. Simultaneous recording of several pills enable to decrease the results variance due to the random component modifying the passage of the pylorus and of the ileo-cecal valve and the peristalsis. Other “Pills” may also be placed at anatomical external landmarks in order to position the detector.

Intake of several pills, for instance one each day, enable to limit the duration of an examination. The pills are spread in the different segments, and thus it is not necessary to wait a full transit time to gather information about all the segments. This is also true if only one segment is to be examined: the probability to have a pill immediately at the right place is higher.

The distance, measured along the skeleton, between several markers enables to differentiate between a general modification of the digestive system activity and an abnormal local activity. For instance, a local decrease in velocity may bring closer the pills in the zone involved, whereas a general decrease in velocity leads to a simultaneous immobilization of all the pills, making clearer the nature of the pathology.

Markers used in the scope of the present invention, as other foreign bodies of similar type passing through the digestive tract, should preferably be recovered after use, because they may contain heavy metals or other substance harmful to the environment.

### Recovery After Use

That is why the present invention also consists in a recovery system for foreign body passing through the digestive tube.

The general principle of the invention is to detect a foreign body in the stools after expelling, and to recover it for appropiate disposal. It is also possible to detect the absence of the foreign body (shown by the detector worn by the subject) to infer its presence in the expelled stools. The recovery device may be part of the detector worn by the subject, fixed on toilets or coming as a hand tool. The later solution is preferable since it enables an accurate localization of the foreign body in the stools.

In the case where the foreign body contains electromagnetic signals emitting means, it is preferable that it continues to emit a signal even if only limited energy is available, for instance once every 10 seconds. It may also emit only once expelled, form instance after detecting a drop in temperature.

The device in question may also be passive or semi-active reemitting a signal following an energy transfer, for instance a “RFID tag” in case of electromagnetic signals.

The foreign body my also contain, in order to be detected, a metallic marker (metal detector, Foucault current), soft ferromagnetic material (measure of reluctance) or hard ferromagnetic i.e. a permanent magnet (measure of magnetic field with for instance: magnetoresistors, Hall sensors, fluxgates, magneto-impedance). In the case of a permanent magnet, the use of a magneto-gradiometer is preferable in order to reduce the influence of the earth magnetic field.

In the case of a hand detector, this one comprises the detection system, a prehension means (for instance pincers) and a disposable part, the only part in contact with the feces. This disposable part may be reversible to be used as trashcan for the foreign body.

The detection system enables to accurately center the prehension system above the foreign body. With this end, the detection system may provide an indication of distance (e.g.: sound signal) and maybe an indication of direction (e.g.: four luminous arrows).

This system is useful for markers used to measure motility by emitting a magnetic field, but it may also be used for any type of foreign body detectable passing trough the digestive tube.

### Encapsulation

Some parameters change the dynamics of the transit. A pill with a high density will have a prolonged residence time in the cecum; a pill with a large diameter will take longer to pass the valves (pylorus, ileo-cecal). The pill may be lightened, for instance, by injection or addition of foam or microballoon resin, If needed, the volume of the pill may be increased with the sole aim to reduce the density. This way, depending on the segment to study or the purpose of the study, different pills may be chosen: For instance, for a gastric emptying time measurement, a small diameter pill is preferred; conversely, for measuring the stomach activity, a large diameter pill is preferred.

A specific shape may be given to the pill in order to direct it in the axis of the digestive tube and in a preferential direction, for instance a long pill. Such a pill, subject to larger rotations, enables a better observation of the wall movements.

A specific shape may be given to the pill in order to accelerate or slow down its forward or backward motions; for instance, with a non constant diameter as for a suppository.

A pill whose shape can be altered may have a larger volume, without being stuck (slowed down) while passing sphincters (e.g. pylorus) or curvature (e.g. duodenum). For instance, a pill coated with elastic foam (closed cell foam) may have a low density and a diameter reduced by digestive tract contractions while passing a sphincter. Note that at ingestion time, the pill may be folded up (compressed foam) and extend only when arriving in a given segment of the digestive tract.

The present invention may also be used for biofeedback; namely a means of influencing the visceral functions. Real time motility data from the system combined with a user friendly graphic display can facilitate biofeedback. This display can be on a pocket PC or via a connection from the detector to a computer with dedicated software.

Instead of being ingested, the pills may be administered as a suppository dedicated to the study the sigmoid colon and rectum motility, or placed in a given segment of the digestive tube, for instance in the stomach (infant).

The device according to the invention permits to quantify digestive tube motility based on motions of one or several markers moving in this tube. Localization of this (these) marker(s) is also known thanks to this device. Advantageously, other functionalities may be combined with the markers according to the invention. For instance, the marker may also transmit images, measure pH and/or pressure, stimulate (or inhibit) motility, change the permeability of the mucus membrane, free a substance or take a swab, or reside in a given segment of the digestive tube. For all these examples, the localization of the pill and the measure of the local mechanical activity is a significant advantage.

### BRIEF DESCRIPTION OF THE DRAWINGS

The invention will be better understood with the description below illustrated by the following figures:

FIG. 1. Schematic representation of an embodiment of the invention.

FIG. 2. Synchronization coil generating a field in several directions, depending on the direction of the current in the coils.

FIG. 3. Data processing flow chart.

FIG. 4. Left: example of rhythmic contractions in the stomach (2.8 cpm) and in the small intestine (9.5 cpm); right: frequency gradient in human.

FIG. 5. Histogram of net motion velocity in human colon. Bimodal distribution showing clearly fast motions (>10 cm/min) and slow motions (<10 cm/min).

FIG. 6, The skeleton, the 3D middle line of the digestive tube represented by a black line, enables to project all dots of the trajectory (here only one dot per minute is displayed) and then to work with a 1D trajectory, i.e. along the skeleton.

FIG. 7. Displacements: spatiotemporal representation of the transit of a pill through the colon; one distinguishes aboral and oral, slow and fast motions.

FIG. 8. Small intestine dynamics: spatiotemporal representation of the transit of a pill through the jejunum and then the ileon; one distinguishes periods of fast progression and of slow progression; the velocity may be a mean value over a given distance (e.g. 5 cm, left curve) or over a given duration (e.g. 3 min).

FIG. 9. Transit vs. Motility. Two groups of patients, clearly separated in this bidimensional representation, would form a unique group if only the transit time was measured. Similarly, patients from group II would merge with healthy subjects if only the local activity was measured.

### DETAILED DESCRIPTION OF PREFERRED EMBODIMENTS

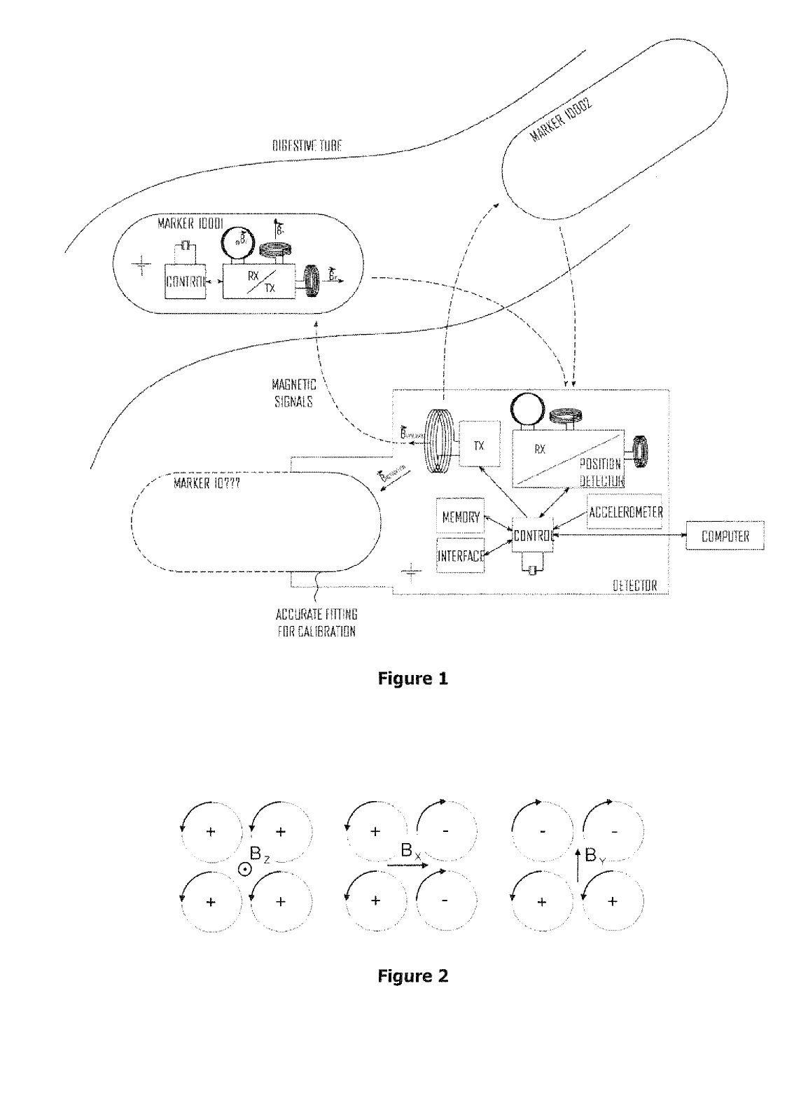

As illustrated in FIG. 1, the device according to the invention includes a pill containing a) three overlapping orthogonal coils, b) a frequency reference, preferably a timing quartz, c) an energy supply, for instance one or two silver oxide batteries and d) electronic circuitry, that is required for microcontroller.

Internal energy supply: any source fitting the circumstances of use of the invention may be employed. Apart from a silver oxide battery, it may also be a supercapacitor (rechargeable), a fuel cell, or electrodes on the surface of the pill (e.g. titanium-platinum) forming an electrochemical cell with the digestive juices. The mechanical energy of the pill motions may also be converted into electric energy or stored in a spring.

The energy may also be transmitted from an external source, for instance by induction.

The coils play the role of emitter and receiver, which enables a bidirectional communication in order to, for instance, synchronizing the pill and the detector for signal demodulation.

In order to increase the amplitude of the emitted signal, a ferromagnetic core may be added.

A unique reference voltage, or reference current, modulated by a signal generated by the electronics excites one after the other the coils. Coils are preferably connected to a capacitance to form a resonant circuit, whose resonant frequency is the working frequency. The working frequency is defined either by a quartz, or a LC oscillator (including the emitting coil), or a RC circuit, for instance inside the microcontroller.

Since the same source is used for all the coils, only one unknown variable is added in case of variation of the source (amplitude of the magnetic dipole). The detector should therefore be made up of a minimum of 4 receiving coils in case of a 2 coils pill, or 3 receiving coils in case of a 3 coils pill, in other words a minimum of 7 equations for 7 unknowns. Redundancy is desired in order to increase the accuracy and the volume where the tracking is possible.

While in emission mode, the pill emits a magnetic signal enabling the tracking of its position. In order to optimally manage the energy available, the pill moves to the emission mode only when the detector requests it and for a limited period of time (for instance a single cycle, one minute or one hour).

The pill emission frequency may be variable, for instance: a) function of the pill motions, especially in the colon where long period of motionlessness alternate with fast motions; or according to the frequency content of these motions; b) according to subject movements, for instance when there are too many artifacts, a high-quality analysis is no longer possible and the sampling frequency can be reduced; or according to the waking state of the subject; c) according to the localization of the pill; d) according to the quality of the recording; e) at predefined moments, for instance the emission may start 5 hours after ingestion to record only colonic segments; f) according to the remaining energy; in particular, to save energy in order to emit for a long period of time (for instance during one month, a few times per minute) enabling to continue locating the pill for recovery purpose or in case of prolonged residence in the digestive tube; g) on operator request (for instance with a button); h) according to other parameters measured by the pill or by the detector.

The pill reverts automatically to the standby mode after emission.

In some variants, for instance in variants a) and f) the pill may define itself the interval between two emissions.

In the simplest case, the pill gets into the emission mode at predefined regular interval (sampling frequency).

Still for a reason of energy management, the emission power—and thus the magnetic momentum—may be changed if necessary. The power is changed, for instance: a) on operator request; b) according to the distance between the pill and the detector, measured either at the pill level (using the amplitude of the synchronization signal) or at the detector level (using the amplitude of the received signals); c) according to the energy still available; d) according to the background noise, measured either at the detector level (using the accuracy given by the algorithm) or at the level of the pill.

In the case where the pill itself decide to vary the amplitude of the magnetic momentum, it may give to the detector a code indicating that the amplitude has changed (for instance using a phase modulation). Preferably, the amplitude takes different predefined values in a list known by the algorithm that calculates the position.

In order to separate signals coming from different emitting coils and from different pills, a multiplexing, either frequential (FDMA), or temporal (TDMA—time division multiple access), or a division with orthogonal codes (CDMA), is used.

Simultaneous emission: in the case of a 2 coils pill, the emission may be in quadrature (90° phase difference of the alternative signal) so simultaneously in the two coils, creating a rotating field, and not successively any more. For a greater number of coils emitting simultaneously (3 coils pill, or several pills), more complex orthogonal signals, such as the ones used in the GPS technique, may be used.

In the case of temporal multiplexing, the multiplexing may be either simply controlled by programming a delayed answer with respect to the synchronization signal from the detector, or using a more sophisticated protocol permitting individual access to each pill. The pills are individually identified either at the production time, or when passing from the turned off mode to the standby mode (activation). A solution also considered is to manage without synchronization signal and therefore to emit with random time interval, being prepared to loose some data.

The detector includes at least one synchronization coil in order to communicate with the pills. If the pill does not include three orthogonal coils, more than one synchronization coil is needed to ensure that the pill receives the signal whatever its orientation (see FIG. 2),

Electrical consumption is a major concern for the marker due to the small size of its batteries. Synchronization of the marker with the detector enables in particular to optimize the period during which the marker listen to the signals from the detector by activating the corresponding interface.

On the detector there is a reference oscillator enabling a synchronous demodulation. The synchronization coil permits to synchronize this oscillator with the one of the pills. These synchronization is completed either before each emission, or often enough to compensate for drift of internal pills oscillators.

Another variant consists in recreating in the detector a reference signal for demodulation, For instance, a PLL may be used to control the reference oscillator of the detector. Note that in this case, the signal may be very weak or zero on some receiving coils; therefore it is preferable to combine signals from different receiving coils. Since the different signals are either in phase or in opposite phase, they can not be directly added (the resultant may be close to zero). There are several solutions, such as: 1) square the signals before adding up, this way a synchronized signal with a doubled frequency is obtained; 2) the preferred solution consists in rectifying the signals and multiplying by the sign of the demodulated signal (+1 when in phase with the reference signal, −1 when in opposite phase); at the beginning, if the PLL is not locked in, an algorithm changes one or more signs until successful locking. Note that the phase noise of the reference signal recreated is a good indicator of the quality of the reception and should be memorized in order to detect artifacts linked to magnetic background.

The detector, or an external part separate, includes an activation coil enabling the transition from the turned off mode to the standby mode through an energy transfer that may be followed by an information transfer (for instance for an identification of the pill). During this activation, the pill is passive, i.e. without current consumption except leakage currents. The voltage induced by the magnetic field generated by the activation coif (pulse, pulse wave or alternative signal) activates an electronic circuit using for instance “Zener zapping” or burning a fuse. In the case of a pill including a microcontroller, the activation circuit may raise an interrupt, generate a reset, activate oscillators or power it on. The activation circuit includes at least an inductance, which may be the emitting coil. It may also include a resonant circuit and/or a rectifier followed by a low-pass filter.

When the pill is activated before the ingestion, different parameters varying from one pill to another can be calibrated: a) placing it in a predefine position: orthogonality and amplitude of the emitted magnetic field can be calibrated; b) whatever the position is: emission frequency, identifier, initialization of a pseudorandom generator (in case of several pills with pseudorandom emission) to ensure a minimum number of superimposed emissions.

Non magnetic alternatives for the activation are: A) a conducting wire, forming a loop external to the pill and connected to the internal electronic circuit, booting up this circuit when it is cut; B) a mechanical impact enables to move or to cause to vibrate an electrical contact, therefore opening or closing an switch; C) if all or part of the coating can be deformed (for instance silicone), a switch can be activated by pressure on the pill; D) the temperature is measured at regular intervals (e.g. every 10s), and the pill is activated if the temperature goes over a threshold (for instance above 30° C., the pill should be kept cool before use); E) two contacts on the pill surface enable to measure the electrical conduction; a change in conduction activates the pill (e.g. by tacking it in the hand); F) measure of a pH change.

The variants D) E) F) permit to activate the pill only when it arrives in the stomach (or the mouth). They may be combined with the dissolving of an external layer of the coating.

In order to transmit information, other than its position, the amplitude, the phase or the frequency of the magnetic momentum may be modulated. The other information to be transmitted may come from:
Inertial sensors (accelerometer, gyroscope); this information may help to quantify the motility index, or may be compared to external sensors for artifacts detection.
Pressure sensor or shape alteration sensor (balloon, flagella); this information enables to evaluate the forces exerted on the pill.
Pressure wave sensor, enabling for instance to record rumbling noise.
Blood detector (e.g, optical detection).
pH sensor.
Bacteria detector; for instance, helicobacter pylon produces ammonia and therefore changes gastric pH; still another example consists in adding lactulose to the coating and then detecting the hydrogen produced (or the lactulose consumption) once the pill in contact with colonic anaerobic bacteria (or the ones that may have colonized the small intestine).
Absence or presence of enzyme; for instance, a non conducting proteinic coating digested in presence of proteases frees surface electrodes, therefore generating an electric signal.
Viscosity sensor (to measure the viscosity of the medium).
Electric conductivity sensor; this information enables for instance to determine the consistency (liquid/solid) of the medium.
Voltage sensor (electro-enterogram, electrogastrogram).

For long recordings, especially for the study of the lower part of the digestive tube, the detector is placed on the abdomen with a harness. The harness includes:
Pockets to hold the detector, including for instance an upper position (epigastric) for recording stomach motility and a lower position (umbilical, hypogastric) for the colon.
The position of the detector may be dorsal (easy access to the lower digestive tract, easy positioning with respect to the anatomy), ventral (more comfortable when lying supine), or even lateral; the drawback of the lateral position is a larger distance detector-pill.

A variant resolving the disadvantage of the lateral position consists in placing to detector, one on each side, both able to calculate independently the position of the pill (so the relative position of the two detectors may be approximate).

The detector includes a user interface that may be a pocket PC or simply buttons and luminous, sound or vibrating indicators. This interface permits for instance to:
The subject may mark certain events; the events may be pain felt by the subject, bowel movement, tacking drug, drinks, meals, or other relevant information.
Show to the subject the sequence of the protocol.
Indicate the presence of the pills, particularly at the end of the recording when the pills have been expelled.
Indicate a weak signal, requiring for instance to change the position of the detector in the harness.
Indicate when too much artifacts occur.
Indicate periods of interest, for instance the subject must stay still (motionless) when the pill reaches a certain localization.
Display in real time the position of the pill, preferably graphically with anatomic landmarks; or display other information useful for biofeedback (motility index, localization, etc.).

Subject movements as well their breathing generates movement artifacts, in other words displacements of the viscera with respect to the detector. In order to detect these artifacts, to filter them out, to evaluate the quality of the recording and to determine the type of activity of the subject, additional sensors are used. These sensors are for instance: inertial sensors (accelerometers, gyrometers), or more simply vibration sensors. The breathing sensor may be for instance a piezoelectric or piezoresistive sensor to measure tensions in the harness. Relative movements between the detector and an anatomical landmark may also be used to detect movement artifacts.

Collected information may be used to modify emission parameters of the pill (sampling frequency for instance) as well as for data processing.

Instead of being ingested, one or several pills may be attached to external anatomical landmarks in order to obtain a frame of reference linked to the subject. Preferably, a reference pill is attached to the xiphoid process. Since the position of these pills is calculated with respect to the detector, it is possible to change the pills trajectory from the detector frame of reference to the anatomical frame of reference. Note that if several landmarks are used (for instance iliac crests and xiphoid process), it will not only be possible to orient and to translate the trajectory but also to scale it with respect to the subject anatomy (homothetic transformation).

Other physiologic measurements relevant for data interpretation may still be collected, for instance:
As already proposed for the pill, a microphone, this time connected to the detector, enables recording of rumbling noise,
Cardiac frequency recording, or electrocardiogram, enables in particular to evaluate the physical activity of the subject, information useful for data interpretation; note that the cardiac frequency variation enables an indirect measure of sympathetic and parasympathetic activity (SNA).
Other information that should be on a diary, such as meals, drinks, glycemia (especially for diabetic patients), can be automatically acquired, for instance: GlucoWatch© system for glycemia, microphone recording swallowing for meal or beverage.

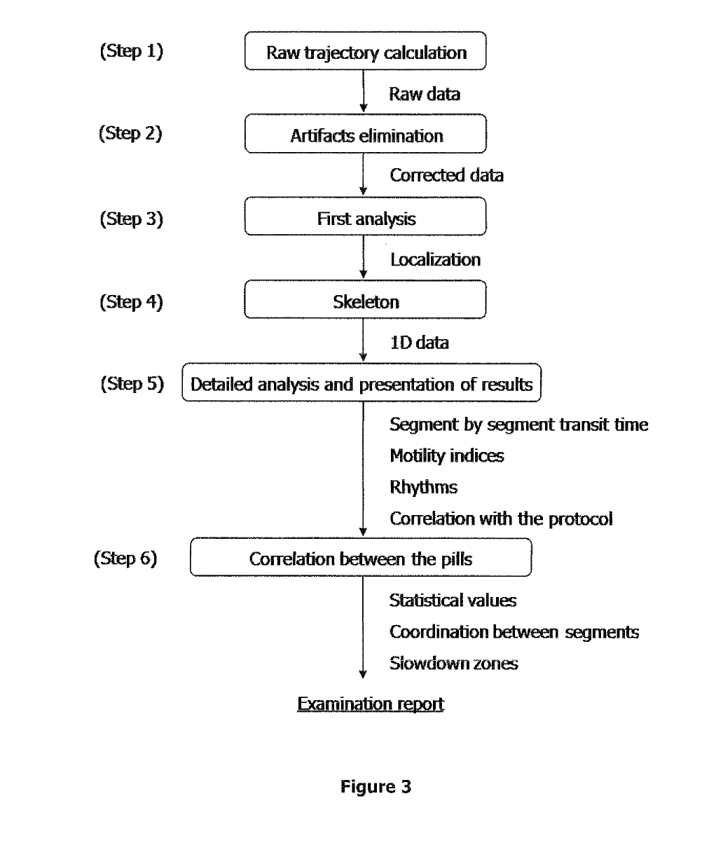

Data Processing Flowchart (FIG. 3).

Position and orientation of the pills are calculated using an algorithm based on the ideal dipole equation.

Removing Artifacts (Step 2)

Artifacts are detected using the following information:
The trajectory itself: detection of non physiologic motions, for instance with too fast velocities or going out of the volume defined by the abdomen; in the absence of external sensor, cardiac and breathing frequency may be obtained from the trajectory itself.
Quality of the reception (background noise) as well as quality of the calculation of the position given by the algorithm (sum of all the noises: background, sensors, etc.).
External sensors, for instance: recording of the breathing and of the movements of the subject, or measure of the position of anatomic landmarks with respect to the detector (pill attached on an anatomic landmark).
In the case when several pills are ingested, the comparison between the trajectory of the different pills may help for artifacts detection: a movement visible simultaneously on all trajectories is probably an artifact.

First Analysis (Step 3)

For data segmentation, i.e. for subdividing and to sort the data according to the segment of the digestive tube involved and also according to the type of displacement, three types of information are used:
Frequency content of the pill motions.
Net motions of the pill.
Position of the pill with respect to anatomic landmarks.

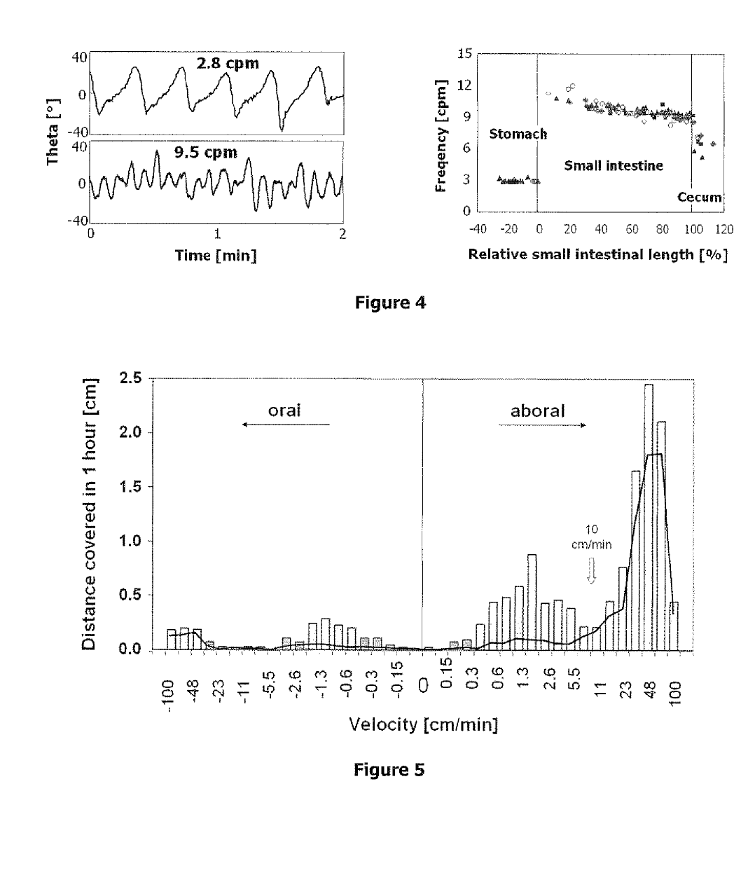

For instance, frequency jump between stomach and duodenum or between ileum and cecum is easily detectable (FIG. 4), making possible a localization based on frequency.

At this stage of the processing, it is difficult to know in which direction (oral or aboral) the pill moves. A preprocessing that removes all the back-and-forth movements is required. For instance, an algorithm may locate when a point of the trajectory go back again through the same position (or relatively close, e.g. less than 5 mm). If the time interval between these two points is less than a chosen threshold, this portion of the trajectory may be defined as a back-and-forth movement (a loop) and then separated from the rest of the trajectory. Other parameters may be taken into account to define a loop, such as a maximum length.

Back-and-forth activity is dealt separately (see step 5)

Once the back-and-forth movements removed, the algorithm locates net displacements, for instance deciding on a lower limit for the distance travelled (e.g. 4 cm) and a lower limit for the mean velocity (e.g. 4 cm/hour). Displacements below these limits are not considered because mainly actifactual (movement artifact not completely filtered out, measurement noise, etc.): such periods are considered as periods without net displacement.

Motions are sorted according to their velocity: slow motions (velocity—mean value over 1 cm—around 1 cm/min in the colon) and fast motions such as colonic mass movements (around 1 cm/s), which can be in the oral or aboral direction (FIG. 5).

Characteristics of these motions will be refined in step 5, after projection onto the skeleton, especially for slow movements.

For instance, a fast movement with a specific shape and direction is characteristic of the passage of the duodenum.

Positioning the detector with respect to the anatomical landmarks enables to translate, rotate and scale the trajectory with respect to the subject anatomy.

It is then possible, for instance with the help of a cross-referencing, to give a prediction of the pill localization,

Skeleton (Step 4)

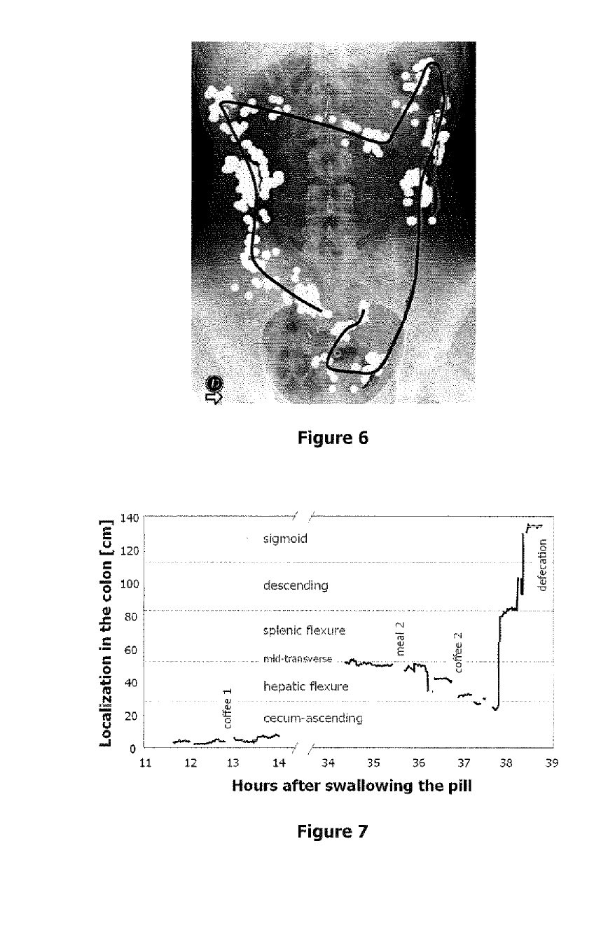

The anatomical trajectory—or middle line of the digestive tube—called here the skeleton (FIG. 6) is calculated for each data segment, using different methods depending on the type of activity: it is relatively trivial to calculate the part of the skeleton corresponding to a colonic mass movement; for very slow or complex displacements, a statistical approach is particularly suitable for identifying forward and backward movements.

It is possible to ask the help of the operator to build the skeleton, to correct errors or to confirm uncertainties, in particular if images of the anatomy are available (e.g. X-rays).

Detailed Analysis and Presentation of the Results (Step 5)

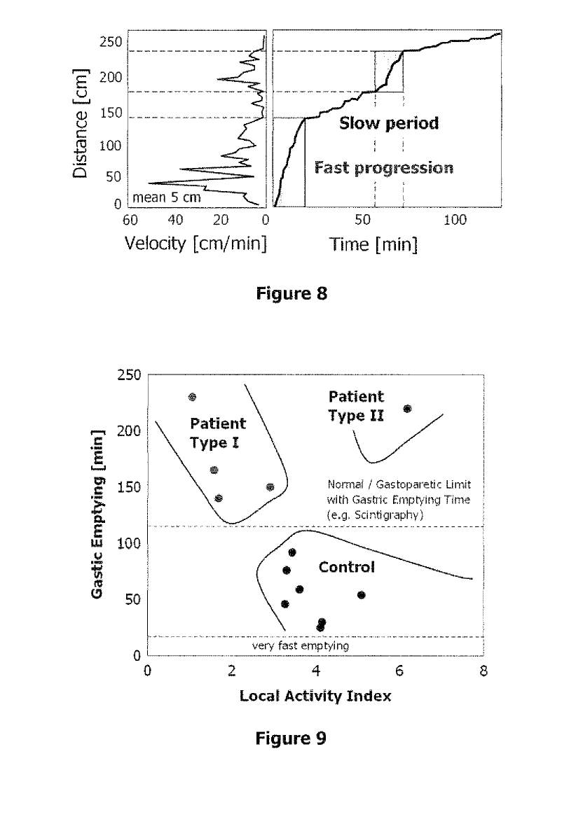

Displacement Index. Once the trajectory is projected onto the skeleton (FIG. 7 and FIG. 8), the algorithm detects net displacements characterized by their direction (oral, aboral), length, velocity and duration. The definition of a net displacement involves the notion of back-and-forth movement and of velocity. When the pill comes back at the same position after a short time (e.g. less than one minute) without moving away too far from this position (e.g. less than 5 cm), it is considered as back-and-forth movement and not as net displacement. When the velocity is too slow (e.g. less than 4 cm/hour) or if the amplitude is too small (e.g. less than 4 cm), it is not considered as a net displacement. The net displacements are then sorted in slow (e.g. <4 cm/min, in the colon) and in fast displacements. Other classifications are also considered, such as long and short displacement (e.g. in the colon, a mass movement is a long (>10 cm) and fast displacement).

Displacement indices can then be calculated for each segment or sub-segment. Displacement indices are basically a function of the distance covered during a certain time or normalized by the length of the segment involved. An index of displacement “total” (caudal+oral), “net caudal” (caudal−oral), “fast” or “slow” can be calculated.

Of course, transit times (sojourn times) segment by segment are also calculated.

Local activity index. Local activity is calculated preferably based on corrected data where the net displacements have been removed (subtracted). Local activity, or trituration, results in back-and-forth movements and rotation of the pill. These movements are characterized by their number, their frequency and the variance of this frequency, their amplitude, shape (sinus, triangle, asymmetric, other predefined shapes), direction and variance of this direction (for instance: in the axis of the skeleton or perpendicular to it).

Therefore, a local activity index “of rotation” and “of translation” can be calculated. The local activity indices are mainly a function of the number, the amplitude and the shape of the movements.

The local activity and the net displacements may be represented with a two-dimensional graphic (FIG. 9) where the separation between different groups clearly appears, which would not be visible with only one or the other information available. Such a graphic also describes well the effect of drugs that affect differently the two parameters, as for instance the morphine which increases the local activity and decreases the net displacement velocity.

Stimulation (or inhibition) of the motility or the modification of the permeability may be optimized, since the localization and the local activity are known thanks to the current invention. The pill may act on the motility and on the permeability through different stimuli:
Electrical.
Thermal.
Mechanical; for instance using vibrations or an inflatable balloon.

If necessary, the power supply may be external, for instance an ultrasound source. Substance release or biopsy (taking samples) may be optimized, since the localization and the local activity are known thanks to the current invention. The pill may release different types of substances:
A drug.
A contrast product.
An antibody.
A photosensitizer to be incorporated into a cancerous tissue, or another substance that can be activated from the outside.

Since the localization and the local activity are known thanks to the current invention, it can be advantageous to immobilize the pill in order to measure the motility or another parameter, to stimulate or to release a substance during a prolonged period in a given segment. The pill may be immobilized by different means, such as:
Mechanically hooked onto the wall.
Immobilized by an external magnetic field, if the pill contains a ferromagnetic material.
By changing the volume of the pill; for example to hold it up in the stomach.

It goes without saying that the invention is not limited to the embodiments described in the present application.

1. A system for gastro-intestinal motility monitoring comprising a solid marker intended to cover all or part of the digestive tract of a living being, a detector comprising means of detecting the position and the orientation of the marker, means of measuring the intestinal motility linked to the detector; the marker and the detector comprising means of emission and means of reception of electromagnetic signals intended to provide a bidirectional communication between the marker and the detector.

2. The system of claim 1, wherein the detector comprises means of communication to initiate at a predefined moment the emission from the marker.

3. The system of claim 2, comprising means of synchronizing the emission from the detector to the marker and the emission from the marker to the detector.

4. The system of claim 1, wherein the means of emission and the means of reception of the marker are made up of the same component.

5. The system of claim 4, wherein the aforementioned component is a coil.

6. The system of claim 5, wherein the marker comprises several coils positioned in different directions.

7. The system of claim 1, wherein the detector comprises means of varying the emission power of the marker.

8. The system of claim 1, wherein the detector comprises an inertial sensor.

9. The system of claim 1, comprising several markers.

10. The system of claim 9, wherein the detector comprises means of communication to initiate at predefined moments the emissions of the markers.

11. The use of a system as defined in claim 1, to analyze the gastro-intestinal motility.

12. The use of claim 11 with several markers and during which each marker is activated at a predefined moment.

13. The use of claim 11, for the localization (for instance stomach, duodenum, jejunum, ileum, cecum, ascending colon, transverse, descending, sigmoid and rectum) according to the type of movement, to the local activity and to the position with respect to an anatomical landmark.

14. The use of claim 11, for data segmentation according to the localization and to the type of movement (for instance, without net displacement, slow movement and fast movement).

15. The use of claim 13, during which the local activity is analyzed by measuring frequencies peculiar to each anatomical segment.

16. The use of claim 12, during which the markers are individually identified before ingestion.
